(19) United States
(12) Patent Application Publication
(10) Pub. No.: US 2025/0388867 A1
(43) Pub. Date: Dec. 25, 2025

(54) COMPOSITION AND METHOD FOR CULTURING ORGANOIDS

(71) Applicant: ORGANOIDSCIENCES, LTD., Gyeonggi-do (KR)

(72) Inventors: Jong-Man YOO, Seongnam-si (KR); Soo-Jung Han, Seongnam-si (KR); Myoung-Ok Nam, Daegu (KR)

(73) Assignee: ORGANOIDSCIENCES, LTD., Gyeonggi-do (KR)

(21) Appl. No.: 19/197,955

(22) Filed: May 2, 2025

Related U.S. Application Data
(63) Continuation-in-part of application No. 16/618,268, filed on Apr. 6, 2020, now Pat. No. 12,319,937.

(30) Foreign Application Priority Data
May 29, 2017 (KR) 10-2017-0065782

(51) Int. Cl. C12N 5/071

(52) U.S. Cl. CPC C12N 5/0679; C12N 5/0697; C12N 2501/999; C12N 2503/04

(57) ABSTRACT

The present invention relates to a composition for culturing organoids and a method for culturing organoids by using the same. Since the present invention includes a compound, which can replace an essential protein component in a conventional medium, consistent stability during organoid culturing can be maintained and culturing can be performed at low cost.

### CROSS-REFERENCE TO THE RELATED APPLICATION

This application is a Rule 53(b) Continuation of U.S. application Ser. No. 16/618,268, filed Nov. 29, 2019, which is a National Stage of International Application No. PCT/KR2018/006057, filed May 29, 2018, claiming priority to Korean Patent Application No. 10-2017-0065782, filed May 29, 2017, the disclosure of which are incorporated herein by reference in their entirety.

### TECHNICAL FIELD

The present disclosure provides a composition for culturing organoids and a method for culturing organoids by using the same.

### BACKGROUND ART

Stem cells are of great interest since they can be used in various fields including the treatment of incurable diseases, disease modeling, tissue or organ transplantation due to the unique features of multi-potency and self-renewal. Recently, it has been found out that when stem cells are cultured in a suitable three-dimensional in vitro environment, a structure similar to the structure of an in vivo organ is formed. This structure formed to have a structure similar to an in vivo organ is referred to as an organoid.

According to organoid related technologies, theoretically, almost all types of organs can be made from only stem cells, and thus organoids are expected to be utilized for various diseases. Organoids may be more effective in testing the stability and efficacy of new drug than cell tissues made in a two-dimensional environment. Additionally, organoids may be transplanted to damaged or underdeveloped organs, and used to improve the condition of the subject. Accordingly, in terms of regenerative medicine, recently, research on organoids is on the rise, and organoids are expected to be widely used in various fields.

Since organoid maintenance and cultivation technology is in the early stage of research and has not been yet established, various researches are to be conducted on what substances are to be added during culturing or how to culture organoids effectively. The addition of R-spondin is essential in a culture medium for ex vivo culturing of organoid. However, it is difficult to isolate and purify R-spondin being a type of protein, and it is also difficult to maintain a consistent level of stability when added to the culture medium. Also, since R-spondin is an expensive substance amounting to about three million KRW per 100 kg, there is a disadvantage that the economic efficiency is poor when mass culturing organoids for use as a therapeutic agent. Therefore, there is an urgent need for an inexpensive and stable R-spondin replacement substance in order to culture clinically applicable organoids.

### Technical Problem

One purpose of the present disclosure is to provide a composition for culturing an organoid. Another purpose of the present disclosure is to provide a method for culturing an organoid.

### Technical Solution

One aspect of the present disclosure is to provide a composition for culturing an organoid comprising a compound of the following chemical formula 1:

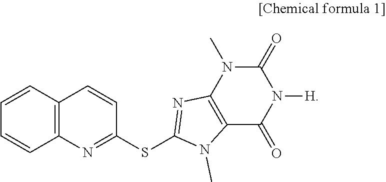

The term “organoid” refers to a cell aggregate made by aggregating and recombining cells isolated from stem cells or organ-derived cells by 3D culture, and may include organoids or cell clusters formed from suspension cell culture. The organoid may also be referred to as a small pseudo-organ, an organ analogue, or a pseudo-organ. Specifically, the organoid includes one or more cell types among various types of cells forming an organ or tissue, and should be able to reproduce the shape and function of the tissue or organ. The organoid may be derived from cells or tissues of human or non-human animal origin. The non-human animals may include, for example, rodents (e.g., mouse, rat), non-human primates, canines, felines, swine, bovines, or other mammals, as well as birds, amphibians, or reptiles. An organoid derived from human cells or tissues is also referred to as a “human organoid”. The cells or tissues may include stem cells, differentiated somatic cells, or heterogeneous cell populations directly isolated from tissue.

The term “organoid culture” includes all actions for producing or maintaining an organoid. For example, it may be differentiating stem cells or cells isolated from particular tissues into tissue or organ cells with specific functions, and/or may be surviving, growing or proliferating an organoid.

The organoid in which the composition of the present disclosure can be used may be, for example, an organoid derived from pluripotent stem cells (PSC-derived organoid), or an organoid derived from adult stem cells (adSC-derived organoid). The pluripotent stem cell may be an embryonic stem cell, a dedifferentiated stem cell, or an induced pluripotent stem cell.

The organoid may be, for example, a stomach organoid, a small intestine organoid, a colon organoid, a liver organoid, a thyroid gland organoid, a lung organoid, a brain organoid, etc. In addition, the organoid may also include, for example, an intestine organoid, a large intestine organoid, a pancreatic organoid, an adenoid organoid, a kidney organoid, a heart organoid, a retinal organoid, a skin organoid, a testis organoid, an ovary organoid, a vascular organoid, a bladder organoid, a salivary gland organoid, a thymus organoid, a prostate organoid, a bone marrow organoid, a lymph node organoid, a muscle organoid, an esophageal organoid, a gallbladder organoid, a bile duct organoid, a nasal cavity organoid, or a cochlear organoid.

The organoid may be a cancer organoid. As used herein, the term “cancer organoid” may be used interchangeably with “tumor organoid” or “tumoroid.” The cancer organoid may include, for example, a pancreatic cancer organoid, colorectal cancer organoid, gastric cancer organoid, liver cancer organoid, lung cancer organoid, breast cancer organoid, prostate cancer organoid, ovarian cancer organoid, bladder cancer organoid, esophageal cancer organoid, brain tumor organoid (e.g., glioblastoma organoid), skin cancer organoid (e.g., melanoma organoid), thyroid cancer organoid, kidney cancer organoid (e.g., renal cell carcinoma organoid), bile duct cancer organoid (e.g., cholangiocarcinoma organoid), cervical cancer organoid, endometrial cancer organoid, testicular cancer organoid, or hematologic cancer organoid (e.g., leukemia organoid, lymphoma organoid, or multiple myeloma organoid).

The culturing composition of the present disclosure is characterized by comprising a compound of the following chemical formula 1:

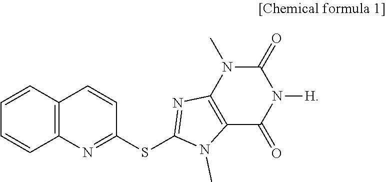

The compound of the chemical formula 1 may be substituted for an activating material of Wnt signal which has been essentially used in conventional organoid culturing. The Wnt signal is important for stem cells to play a normal role, and thus the activating material of Wnt signal is necessarily required to be added to an organoid culture. Currently, Wnt3a or R-spondin is used as the activating material of Wnt signal in order to culture organoids. In the present disclosure, the compound of chemical formula 1 can replace the Wnt3a or R-spondin when culturing organoids. In one embodiment, when organoids are cultured in a culture medium containing the compound of chemical formula 1 instead of R-spondin, their morphological characteristics, growth efficiency and marker's expression level showed similar to those of the organoids cultured in the medium containing R-spondin. Therefore, the composition may not comprise Wnt3a or R-spondin, or may comprise less than the usual concentration of Wnt3a or R-spondin which has been generally used in organoid culturing. The concentration of the compound of chemical formula 1 comprised in the composition may be, for example, 5 to 200 μM, 6.25 to 200 μM, 6.25 to 100 μM, 10 to 100 μM, 10 to 75 μM, 20 to 75 μM, 25 to 75 μM, 20 to 50 μM, or 25 to 50 μM with respect to the total volume or amount of the composition. Preferably, the concentration of the compound of chemical formula 1 may be 20 to 75 μM, 25 to 75 μM, 20 to 50 μM, or 25 to 50 μM, and more preferably may be 25 to 75 μM, or 25 to 50 M. Most preferably, the concentration of the compound of chemical formula 1 may be 50 M.

The composition may be a basal culture medium for culturing organoids in which the compound of chemical formula 1 is comprised. The term “culture media” or “culture medium” means a medium which enables support for in vitro cell growth and survival, and includes all conventional culture media used in the art as being suitable for culturing cells. The culture medium and culture condition may be selected depending on the type of cultured cells. As examples for basal medium for culturing cells, Dulbecco's modified eagle's medium (DMEM), minimal essential medium (MEM), basal medium eagle (BME), RPMI1640, F-10, F-12, Glasgow's minimal essential medium (GMEM), Iscove's modified Dulbecco's medium, etc. may be used, and antibiotics such as penicillin-streptomycin or supplements, etc. may be further added as needed.

The composition of the present disclosure may further comprise an ingredient necessary for signaling pathway or organoid formation of stem cells. Specifically, the composition may further comprise one or more selected from the group consisting of epidermal growth factors (EGF), noggin, thiazovivin, CHIR99021 and a pharmaceutically acceptable salt of CHIR99021. The CHIR99021 refers to 6-[[2-[[4-(2,4-dichlorophenyl)-5-(5-methyl-1H-imidazole-2-yl)pyrimidin-2-yl]amino]ethyl]amino]-3-pyridine carbonitrile (CAS No.: 252917-06-9), which is a compound of the following chemical formula 2:

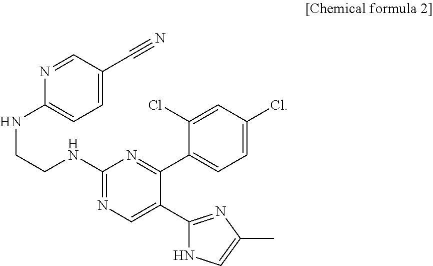 [SMILES: Cc1c[nH]c(-c2cnc(NCCNc3ccc(C#N)cn3)nc2-c2ccc(Cl)cc2Cl)n1]

Another aspect of the present disclosure is to provide a method for culturing an organoid, comprising culturing cells in a composition containing the compound of the following chemical formula 1:

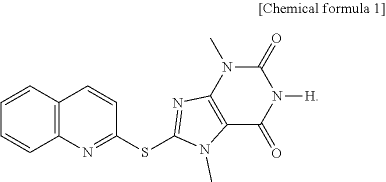

In the method for culturing organoid according to the present disclosure, the same terms as used in the composition for culturing organoid according to one aspect are used as mentioned in the above composition, unless otherwise specified.

The method for culturing organoid according to the present disclosure may comprise the steps of contacting a cell with the compound of chemical formula 1, and culturing the cell. The cell may be a stem cell, a population of stem cells, a cell differentiated from stem cells, or an isolated tissue fragment. Preferably, the cell may be an adult stem cell, and more preferably may be a cell included in or derived from small intestine crypt.

The concentration of the compound of chemical formula 1 comprised in the composition may be, for example, 5 to 200 μM, 6.25 to 200 μM, 6.25 to 100 μM, 10 to 100 μM, 10 to 75 μM, 20 to 75 μM, 25 to 75 μM, 20 to 50 μM, or 25 to 50 μM with respect to the total volume of the composition. Preferably, the concentration of the compound of chemical formula 1 may be 20 to 75 μM, 25 to 75 μM, 20 to 50 μM, or 25 to 50 μM, and more preferably may be 25 to 75 μM, or 25 to 50 M. Most preferably, the concentration of the compound of chemical formula 1 may be 50 M.

The composition of the present disclosure may further comprise an ingredient necessary for signaling pathway or organoid formation of stem cells. Specifically, the composition may further comprise one or more selected from the group consisting of epidermal growth factors (EGF), noggin, thiazovivin, CHIR99021 and a pharmaceutically acceptable salt of CHIR99021. The CHIR99021 refers to 6-[[2-[[4-(2,4-dichlorophenyl)-5-(5-methyl-1H-imidazole-2-yl)pyrimidin-2-yl]amino]ethyl]amino]-3-pyridine carbonitrile (CAS No.: 252917-06-9), which is a compound of the following chemical formula 2:

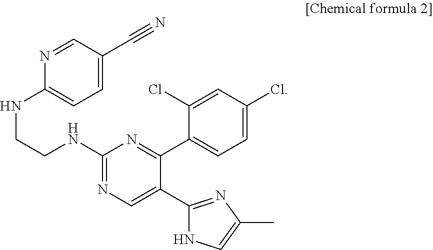 [SMILES: Cc1c[nH]c(-c2cnc(NCCNc3ccc(C#N)cn3)nc2-c2ccc(Cl)cc2Cl)n1]

In the culturing method, the composition may not comprise Wnt3a or R-spondin, or may comprise less than the usual concentration of Wnt3a or R-spondin which has been generally used in organoid culturing.

In the composition for culturing organoid and the culturing method according to the present disclosure, it comprises a compound that can replace the protein ingredient essentially added to the conventional culture medium. Accordingly, it is possible to maintain a consistent stability when culturing organoids, and organoids can be cultured at a low cost. Therefore, the present disclosure may be utilized for mass culturing of organoids for use in the development of a therapeutic agent.

One embodiment of the present invention provides a method for culturing an organoid using a compound of Chemical formula 1; a composition comprising the compound; a composition supplemented with the compound; or a culture medium containing the compound or the composition.

In the present invention, the compound of Chemical formula 1 serves as the essential and sufficient component for culturing organoids. The compound may be added as a supplement to a basic culture medium, such as Advanced DMEM/F12, without requiring the addition of any other exogenous growth factors, pathway modulators, or supplements. When added to the basic medium, the compound alone enables the initiation, growth, and maintenance of organoids. Accordingly, the compound of Chemical formula 1 is considered a functionally active component that does not rely on the co-presence of conventional supplementary components to exert its organoid-supporting effects.

An organoid cultured according to the method of the present invention may be administered or transplanted into a subject. As used herein, the term “subject” refers to a biological recipient that may benefit from the functional restoration, replacement, or evaluation of tissue or organ function through organoid-based transplantation or assay. The subject may include humans or non-human animals, including, but not limited to, rodents (e.g., mouse, rat), rabbits, dogs, cats, pigs, sheep, goats, horses, non-human primates (e.g., cynomolgus monkey, rhesus macaque), or other mammalian species commonly used in preclinical or veterinary models. The subject may be a patient with an existing disease or disorder, or an individual at risk of developing such conditions, including, but not limited to, inflammatory bowel disease (IBD), such as Crohn's disease and ulcerative colitis; autoimmune or immune-mediated diseases affecting the gastrointestinal tract; salivary gland disorders, such as Sjogren's syndrome or radiation-induced xerostomia; tonsillar hypertrophy or recurrent tonsillitis; pancreatic insufficiency; gastrointestinal neoplasms, including colorectal adenomas and early-stage cancers; epithelial dysplasia or metaplasia; post-surgical tissue reconstruction; intestinal ischemia-reperfusion injury; and genetic disorders involving epithelial stem cell dysfunction.

The organoid obtained by the present method may also be used in non-therapeutic applications, including high-throughput screening and safety assessments of bioactive materials. Such applications include, without limitation, the evaluation of pharmaceutical agents (including small molecules, biologics, and peptides), cosmetic ingredients (e.g., anti-aging or barrier-modulating compounds), and functional food substances (e.g., probiotics, polyphenols, or gut-active nutrients). The evaluation may include endpoints such as morphological integrity; viability and proliferation rate; barrier function (e.g., transepithelial electrical resistance, TEER); gene or protein expression markers; immune or cytokine response profiles; and cytotoxicity (e.g., lactate dehydrogenase (LDH) release, apoptosis assays).

In a specific embodiment, the organoid is a cancer organoid and may be derived from patient tumor samples or cancer cell lines. In such cases, the present invention provides a method for evaluating the efficacy of an anti-cancer agent, comprising administering the agent to the cancer organoid and assessing tumor-specific endpoints (e.g., tumor size reduction, apoptosis induction, marker expression changes); and a method for screening anticancer agents, comprising contacting the cancer organoid with test candidates and evaluating therapeutic potential using defined molecular or phenotypic assays. The cancer organoid may be derived from a wide range of malignancies, including, but not limited to, pancreatic, colorectal, gastric, hepatic, pulmonary, breast, prostate, ovarian, thyroid, renal, bile duct, esophageal, cervical, endometrial, testicular, skin (e.g., melanoma), and brain tumors (e.g., glioblastoma), as well as hematological malignancies such as leukemia, lymphoma, and multiple myeloma.

The articles “a” and “an” are used herein to refer to one or to more than one (i.e. to at least one) of the grammatical object of the article. Byway of example, “an ingredient” means one ingredient or more than one ingredient.

### BRIEF DESCRIPTION OF FIGURES

The patent or application file contains at least one drawing executed in color. Copies of this patent or patent application publication with color drawing will be provided by the Office upon request and payment of the necessary fee.

FIG. 1A is a photograph of one of 105 96-well plates used for compound screening. The six wells in the leftmost column were used as a positive control group, the six wells in the rightmost column were used as a negative control group, and the entire ten columns in the middle were used as an experimental group.

FIG. 1B shows the result of selecting a second candidate material using the total small intestinal organoid number (viability factor) and the budding small intestinal organoid number (budding factor).

FIG. 1C is an optical micrograph of small intestinal organoid grown in a culture solution comprising a compound determined to be significant as a result of screening.

FIG. 2A is an optical micrograph of mouse intestinal crypt treated with RS-246204 and cultured for 4 days, where ENR is a positive control group (including R-spondin), EN is a negative control group, and EN+RS-246204 is an experimental group.

FIG. 2B is a graph showing the result of WST analysis for confirming the survival rate of small intestinal organoids according to RS-246204 concentration. The Y axis is a value obtained by converting the WST activity value into percentage, and a negative control group is set to 100%.

FIG. 2C is a graph showing the number of different forms of organoids after growth of small intestinal organoids according to RS-246204 concentration. The Y axis is the number of organoids present in the well. Budding organoids refer to budded organoids, and non-budding organoids refer to living organoids which have not been budded. Total viable organoids refer to a sum of the number of budding organoids and non-budding organoids.

FIG. 2D is a graph measuring the average length of organoid circumference by date. In the graph, DIV refers to day in vitro, the Y axis is the percentage value of the average organoid circumference, and DIV −1 is set to 100%.

FIG. 2E is an optical micrograph of subculturing after primarily culturing small intestinal organoids under ENR and EN+RS246204 conditions, respectively.

FIG. 3A is a photograph showing the result of electrophoresis after RT-PCR using RNA extracted from small intestinal organoids cultured under ENR and EN+RS246204 conditions, respectively.

FIG. 3B is a graph showing qRT-PCR results using RNA extracted from small intestinal organoids cultured under ENR and EN+RS246204 conditions, respectively.

FIG. 3C shows an immunofluorescence staining results of small intestinal organoids cultured for 4 days under ENR and EN+RS246204 conditions. Hoechst (blue) represents the nucleus, and Alexa594 (red) represents the fluorescence for each antibody.

FIG. 4 is an optical micrograph of inducing forskolin stimulation after culturing small intestinal organoids for 4 days under ENR and EN+RS246204 conditions, respectively.

FIG. 5 shows the result of formation and proliferation of human large intestinal organoids when culturing using EGF (E), Noggin (N), R-spondin (R), and/or RS-246204.

FIG. 6 shows the result of formation and proliferation of human large intestinal organoids when culturing using RS-246204 (0, 2.5, 5, 50 or 100 PM).

FIG. 7 shows the result of formation and proliferation of large intestinal organoids when culturing using R-spondin, or 20 μM RS-246204. The “R-spondin (−)” condition means that it does not comprise either R-spondin or RS-246204.

FIG. 8 shows the result of formation and proliferation of salivary organoids when culturing using R-spondin, or 1.25 μM, 2.5 μM or 5 μM RS-246204.

FIG. 9 shows the result of formation and proliferation of adenoid organoids when culturing using R-spondin, or 1 μM, 2 μM or 5 μM RS-246204.

FIG. 10 shows the result of formation and proliferation of pancreatic cancer organoids when culturing using R-spondin, or 30 μM RS-246204.

### EXAMPLES

In the following, exemplary embodiments of the inventive concept will be explained in further detail with reference to examples. However, the following examples are meant to exemplify the present invention, and the scope of the invention is not restricted by these examples.

### Example 1. Isolation of Mouse Small Intestinal Crypt

Small intestinal crypts were isolated from a mouse to be used in experiments for preparing and culturing small intestinal organoids. Specifically, the small intestine was isolated after killing a 5-7 weeks old C57BL/6 mouse weighing 20-25 g by cervical vertebrae dislocation. The small intestine was cut longitudinally from a proximal end to a distal end, and also laterally cut into pieces of about 5 mm length. The piece of small intestine obtained was washed with ice-cooled Dulbecco's phosphate-buffered saline (DPBS) until the supernatant liquid was sufficiently clear. Then, the crypts were isolated by treating with a Gentle Cell Dissociation Reagent (StemCell Technologies, Cambridge, MA), and filtering with a cell strainer.

### Example 2. Screening of Compound Library Using Organoid Proliferation Measurement

Compounds which can replace R-spondin in organoid cultures were screened for 8,364 types of compounds included in a representative library of the Korea Chemical Bank.

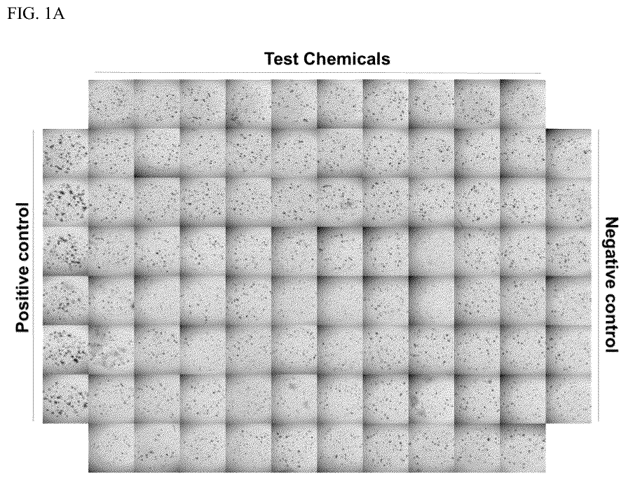

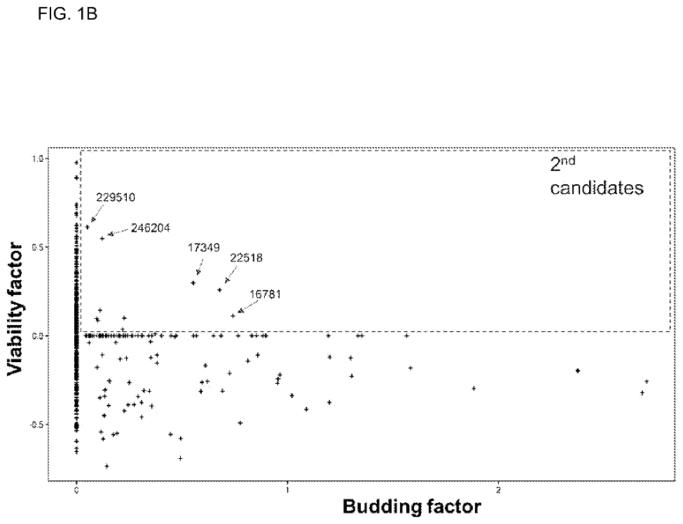

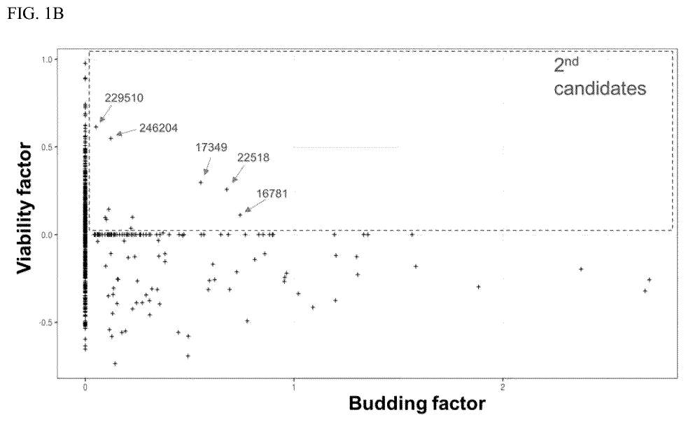

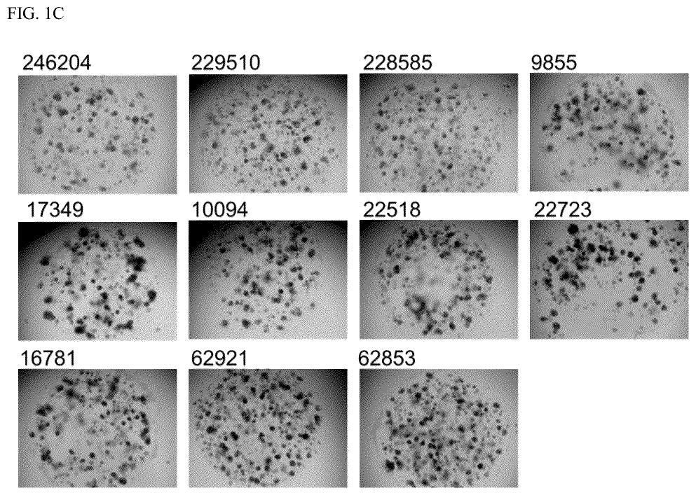

The small intestinal crypts derived from the small intestine of a 7-week-old C57BL/6 mouse isolated in Example 1 were mixed in Matrigel, and placed in each well of a 96-well plate. An experimental group of eighty wells was used per 96-well plate, three wells were used for each of the positive control group and the negative control group, and three wells were used for each of the positive control group and the negative control group including 0.5% of DMSO. The negative control group used an EN culture solution which does not contain R-spondin nor compounds (composition: advanced DMEM/F-12, Hepes buffer solution, GLUTAMAX-I SUPPLEMENT, penicillin-streptomycin solution, N-acetyl-L-cysteine, B-27 serum-free supplement, N-2 supplement, animal-free recombinant murine EGF, recombinant murine noggin, CHIR99021, thiazovivin). The positive control group used the EN culture solution in which 10% of R-spondin is contained. Hereinafter, the EN culture solution containing 10% of R-spondin is referred to as ENR culture solution. As the experimental group, 8,364 different types of compounds were added to the EN culture solution at a concentration of 50 μM, respectively. The compounds used for screening were treated immediately after completing the polymerization of matrigel and crypt, and was applied without replacement for 4 days immediately after isolating crypts. After keeping in 37° C. humidification incubator (5% CO₂) for 4 days, the cultured organoids were observed and optical microscope photos were taken for analysis (see FIG. 1A).

The number of living organoids, the number of budding organoids, and the circumference of each organoid were measured using the optical microscope photos. For counting, a cell counter plugin of Image J Software was used, and for measuring the circumference, a free curve tool of Dixi eXcope software was used. The compounds were ranked based on the measured values, and then 295 candidate compounds were selected to be used in a second screening. The 295 candidate compounds for second screening were re-examined in the same manner as first screening. After that, 21 candidates were selected by collecting and ranking the results of the first and second screening. After performing a third screening for the 21 candidate materials in the same manner, all three results were collected to finally select seven candidate compounds (see FIG. 1B).

Among the final seven candidate compounds, the compound of the following chemical formula 1 (Compound Library No.: STK611777) showed the highest growth of small intestinal organoids among the candidate materials, and it was observed from numerical data and visual confirmation that said compound could grow and maintain organoids most similar to R-spondin (see FIG. 1C):

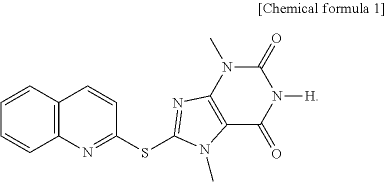

The compound is referred to as “RS-246204.”

### Example 3: Small Intestinal Organoid Culture Effect of RS-246204

### 3.1. Culture Effect According to Concentration of Compound

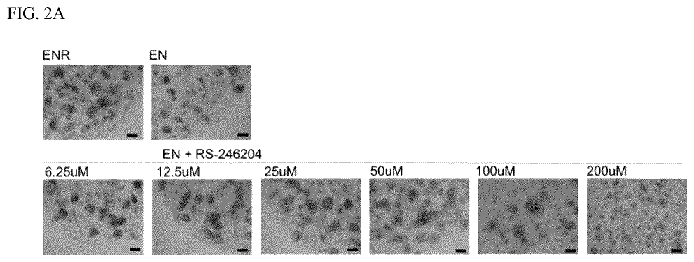

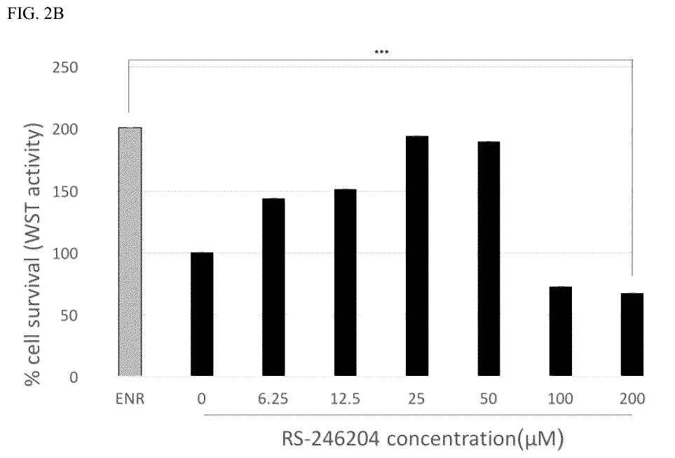

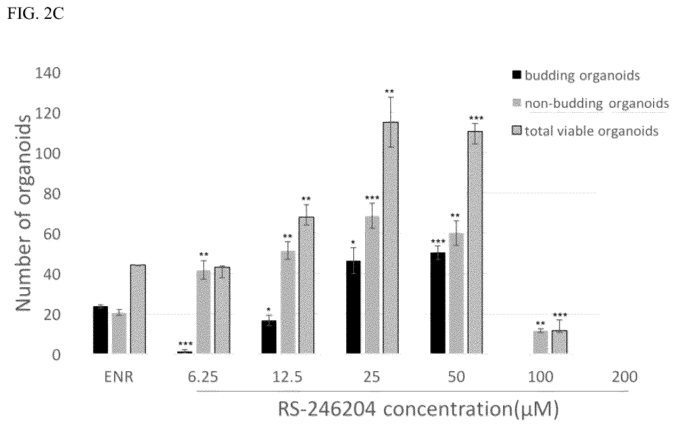

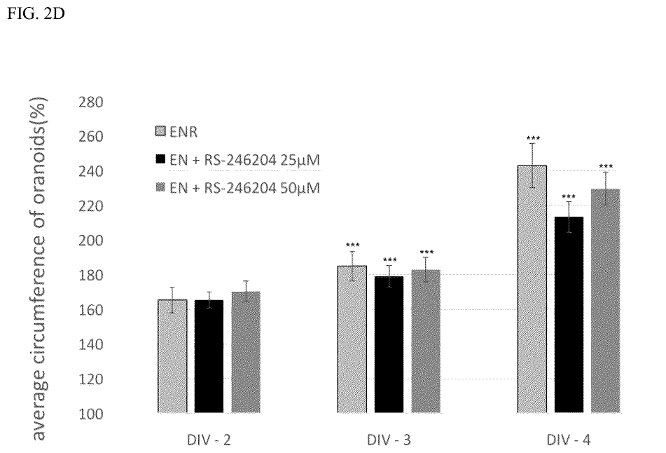

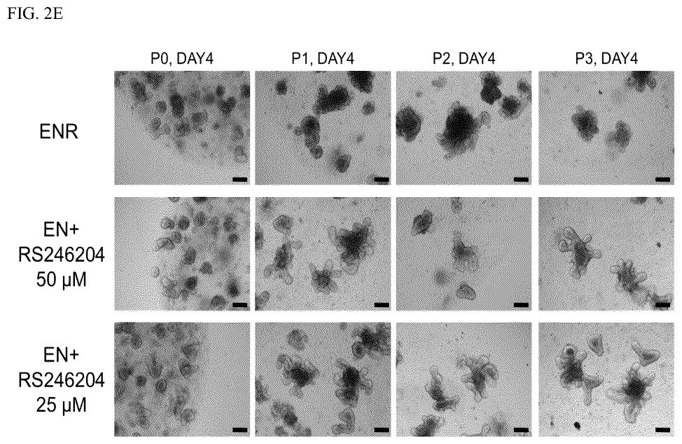

An optimal concentration on the small intestinal organoids culture effect of RS-246204 was examined. RS-246204 was added to the culture solution of the small intestinal crypts, respectively, at a final concentration of 6.25 μM, 12.5 μM, 25 μM, 50 μM, 100 μM, and 200 μM, and then was incubated in the culture medium for 4 days. After 4 days, it was observed that the small intestinal organoids grown in the culture solution containing 25 μM and 50 M of RS-246024 showed similar morphology and growth to the small intestinal organoids grown of the ENR culture solution (see FIG. 2A).

### 3.2. WST Analysis

For more accurate confirmation, RS-246204 was treated at the same concentration, and after 4 days, 10 μl of WST was added to each well. WST is a tetrazolium salt which reacts with dehydrogenase to produce formazan, thereby causing the culture solution to become orange color. The dehydrogenase is an enzyme which exists only in a living cell, and thus it is possible to check the survival rate of cell when treated with WST. 3 hours after adding WST, only the culture solution was taken, and the absorbance was measured at 450 nm. When the survival rate in the EN culture solution, which is a negative control group, is set to 100%, the culture solution containing 25 μM and 50 μM of RS-246204 showed an organoid survival rate similar to that in the ENR culture solution, which is a positive control group (see FIG. 2B).

As a result, it was observed that when the small intestinal organoids were cultured using the isolated crypts, the small intestinal organoids grown in the culture solution in which R-spondin was replaced with RS-246204 grew in a manner similar to the small intestinal organoids grown in the conventional culture solution containing R-spondin.

### 3.3. Budding Characteristics and Appearance Analysis

The morphological characteristic of the small intestinal organoids is that they grow while budding. In order to check that the budding rate of the small intestinal organoids grown in the RS-246204-added culture solution (hereinafter, RS-246204 culture solution) is similar to that grown in ENR culture solution, the total number of organoids, the number of budding organoids, and the number of non-budding organoids were counted in the wells culturing for 4 days. The rate of budding organoids and non-budding organoids grown in the culture solution containing 50 μM of RS-246204 is about 1:1, which is similar to the rate of organoids grown in the ENR culture solution (see FIG. 2C). It was observed that the rate of non-budding organoids increased at a concentration lower than 50 μM, and most organoids were killed at a concentration higher than 50 M.

In order to check whether the growth efficiency of the small intestinal organoids grown in the RS-246204 culture solution has a difference, the small intestinal organoids cultured in each culture solution were photographed by an optical microscope every day. The circumference of the individual organoid according to the condition of culture medium and date was measured, and converted into percentage. As a result, it was observed that not only the growth efficiency but also the increase rate of the circumference according to date were also similarly observed (see FIG. 2D).

### 3.4. Subculture

It was confirmed that it is possible to subculture small intestinal organoids grown in RS-246204 culture solution in the same manner as small intestinal organoids grown in ENR culture solution, and it was observed that the growth and maintenance were also possible even after the subculture (see FIG. 2E). The crypts cultured in a culture solution which does not comprise R-spondin or RS-246204 could not be cultured to the small intestinal organoids.

### Example 4. Analysis of Gene Expression of Small Intestinal Organoids Cultured by RS-246204

### 4.1. RT-PCR Analysis

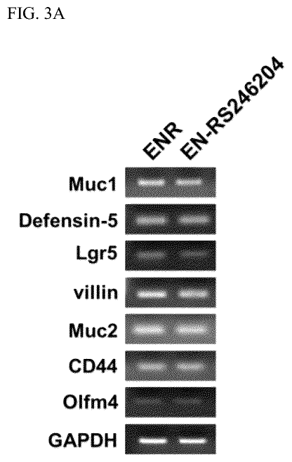

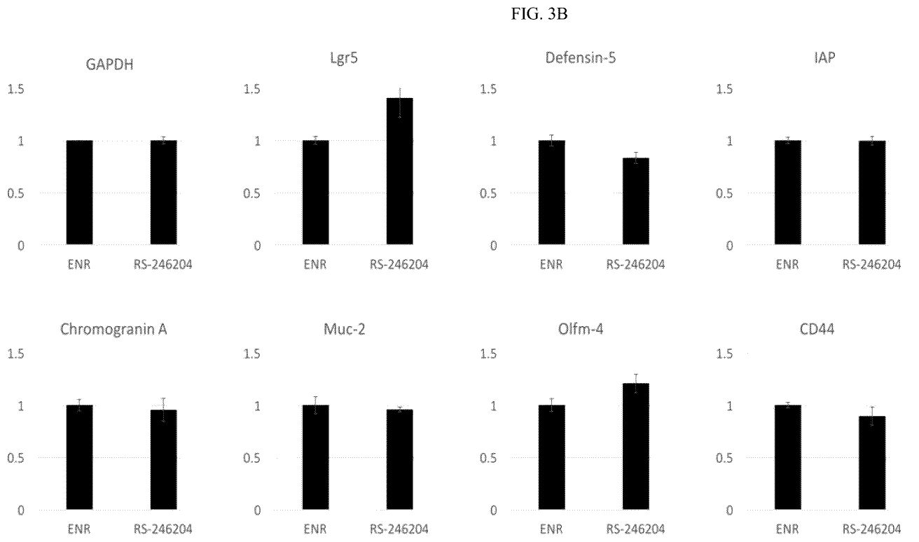

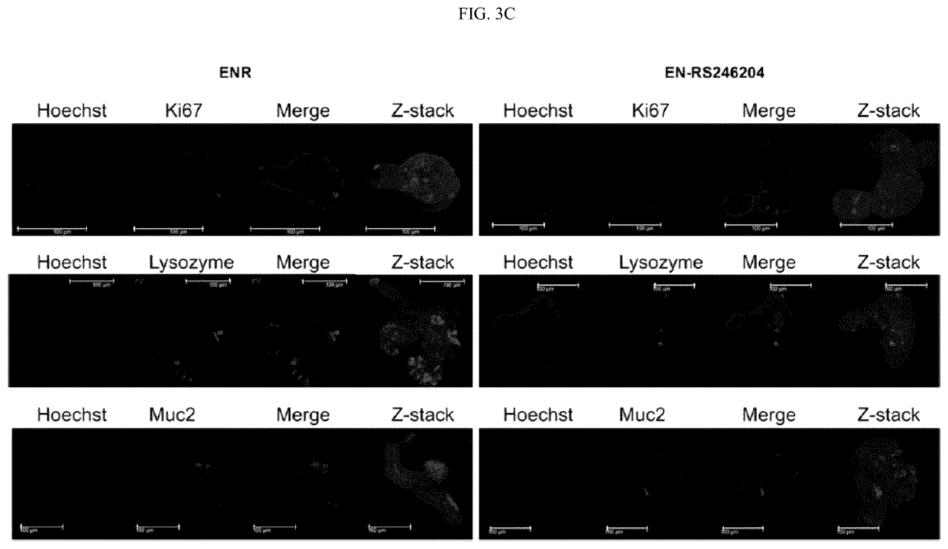

In order to confirm whether lineage markers specific to the small intestinal organoids are expressed when cultured with RS-246204, RNA analysis was performed. The small intestinal organoids cultured in ENR and RS-246204 culture solutions, respectively, for 4 days were collected. RNA was extracted and cDNA was synthesized, and then RT-PCR was performed. As a marker for intestinal stem cells, goblet cells, paneth cells, enteroendocrine cells and enterocyte, RT-PCR was performed using RNA primers for Lgr5, muc-1 and muc-2, defesing-5, chromogranin A (ChgA), and villin, respectively. As a result, it was confirmed that all these cells were present in the small intestinal organoid cultured by RS-246204, and that Olfactomedin-4 (Olfm4) and CD44, which are genes located downstream of Lgr5 signaling, were also expressed (see FIG. 3A).

For quantitative analysis, qRT-PCR analysis was performed. AccuPower 2X Greenstar qPCR MasterMix (Bioneer) and Thermal Cycler Dice® Real Time System III (Takara, Japan) were used, and the reaction was performed for 10 seconds at 95° C. (denaturation), 15 seconds at 57° C. (annealing), and 20 seconds at 72° C. (extension). The RNA primers excluded Muc1 among those used in the RT-PCR test, changed the enterocyte marker from villin to Intestinal Alkaline Phosphatase (IAP), and used the same sequence for the rest. The qRT-PCR results showed that the relative amount of markers expressed showed little difference between the ENR and RS-246204 culture solutions (see FIG. 3B).

### 4.2. Immunofluorescence Analysis

In addition, immunofluorescence staining was performed on the small intestinal organoids cultured in the ENR and RS-246204 culture solutions to confirm the expression of Muc-2, lysozyme, and Ki67, which is a marker of proliferating cells (see FIG. 3C).

### Example 5. Function Maintenance of Small Intestinal Organoids Cultured by STK611777

In order to check whether functions of small intestinal epithelial cells are maintained in the small intestinal organoids cultured using the RS-246204 culture solution, CFTR agonist Forskolin analysis was performed. The forskolin is a compound which stimulates ion channels to open, thereby promoting the release of moisture. In case of small intestinal organoids, moisture gathers into the lumen by the forskolin stimulation and changes into a large spherical shape, thereby confirming the maintenance of the function of epithelial cells. After culturing for 4 days in RS-246204 culture solution and ENR culture solution, respectively, it was replaced with a culture solution to which forskolin was added at a concentration of 5 μM. Photographs were taken by an optical microscope at an interval of 10 minutes for 1 hour immediately after the replacement. Based on the optical micrographs, the free curve tool of Dixi eXcope (Korea) program was used to measure the circumference of each organoid per hour and analyze the change in circumference.

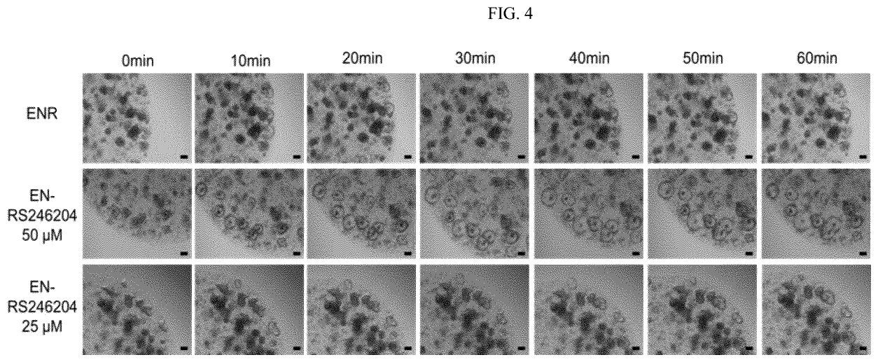

As a result, it was observed that the small intestinal organoids grown in RS-246204 culture solution performed well as epithelial cells, and showed a similar change in circumference to those grown in ENR culture medium (see FIG. 4).

### Example 6. Formation of Large Intestinal Organoids

Large intestinal tissue was isolated from a human patient during surgery and used in experiments for forming organoids. The tissue was cut longitudinally from a proximal end to a distal end, and also laterally cut into pieces of about 5 mm length. The tissue pieces, which were isolated crypts, were washed with ice-cooled Dulbecco's phosphate-buffered saline (DPBS) until the supernatant liquid was sufficiently clear. Then, the crypts were isolated by treating with a Gentle Cell Dissociation Reagent (StemCell Technologies, Cambridge, MA), and filtering with a cell strainer. The crypts were mixed in collagen gel, and placed in each well of a well plate with the composition comprising the compound of the chemical formula 1 (namely, RS-246204). After keeping in 37° C. humidification incubator (5% CO₂) for 5 days, the formed organoids were observed, and optical microscope photos were taken for analysis.

The control and experimental groups were classified as follows depending on the components of the medium composition used.

### (1) Control Group:

ENR group: Basal media supplemented with EGF (E), Noggin (N) and R-spondin (R) was used to form human large intestinal organoids.
EN+RS-246204 group: Basal media supplemented with EGF (E), Noggin (N) and RS-246204 (25 or 50 μM) was used to form human large intestinal organoids.

### (2) Experimental Group:

RS-246204 group: Basal media supplemented with RS-246204 (25 or 50 μM) was used to form human large intestinal organoids.

Noggin, thiazovivin, CHIR99021, and a pharmaceutically acceptable salt of CHIR99021 was not used in both the control and experimental groups.

### Results:

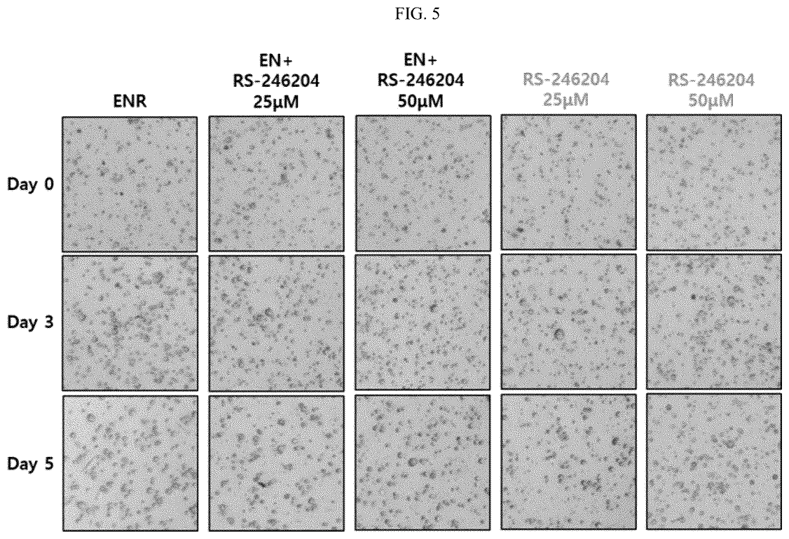

Large intestinal organoids formed from crypts in each group have the characteristics below (see FIG. 5).
(1) ENR group (the control group): When crypts were cultured in the basal media supplemented with EGF, Noggin, and R-spondin, human intestinal organoids were formed.
(2) EN+RS-246204 group (the control group): When crypts were cultured in the basal media supplemented with EGF, Noggin, and RS-246204 (specifically, R-spondin is replaced by RS-246204), human intestinal organoids were formed.
(3) RS-246204 group (the experimental group): When crypts were cultured in the basal media supplemented with RS246204, human intestinal organoids were formed. Comparing the quantity of organoid calculated from the confluency, there is no significant difference with that in the ENR or EN+RS-246204 groups.

### Example 7. Formation of Large Intestinal Organoids

Large intestinal tissue was isolated from a human patient during surgery and used in experiments for forming organoids. The tissue was cut longitudinally from a proximal end to a distal end, and also laterally cut into pieces of about 5 mm length. The tissue pieces, which were isolated crypts, were washed with ice-cooled Dulbecco's phosphate-buffered saline (DPBS) until the supernatant liquid was sufficiently clear. Then, the crypts were isolated by treating with a Gentle Cell Dissociation Reagent (StemCell Technologies, Cambridge, MA), and filtering with a cell strainer. The crypts were mixed in collagen gel, and placed in each well of a well plate with the composition comprising the compound of the chemical formula 1 (namely, RS-246204). After keeping in a 37° C. humidification incubator (5% CO₂) for 5 days, the formed organoids were observed, optical microscope photos were taken for analysis, and organoid counts were analyzed using Leopard iX software.

The control and experimental groups were classified as follows depending on the components of the medium composition used.

### (1) Control Group:

RS 0 μM group: Basal media (Advanced DMEM/F12 media) not supplemented with RS-246204 (also referred to as “RS”) was used to form human large intestinal organoids.
RS 2.5 μM group: Basal media (Advanced DMEM/F12 media) supplemented with RS-246204 2.5 μM was used to form human large intestinal organoids.

### (2) Experimental Group:

RS 5 μM group: Basal media (Advanced DMEM/F12 media) supplemented with RS-246204 5, 50 or 100 μM was used to form human large intestinal organoids.
RS 50 μM group: Basal media (Advanced DMEM/F12 media) supplemented with RS-246204 50 μM was used to form human large intestinal organoids.
RS 100 μM group: Basal media (Advanced DMEM/F12 media) supplemented with RS-246204 100 μM was used to form human large intestinal organoids.

### Results:

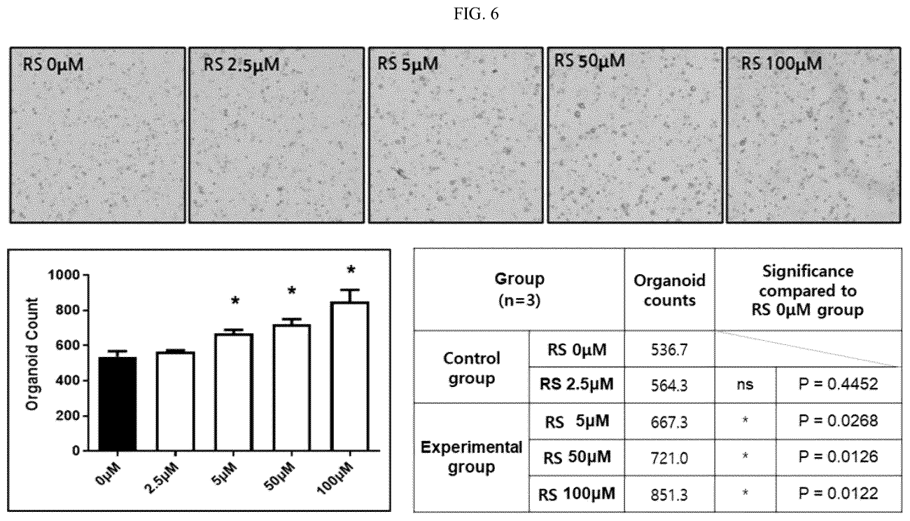

Large intestinal organoids formed from crypts in each group have the characteristics below (see FIG. 6).
(1) RS 0 μM group (the control group): When crypts were cultured in the basal media not supplemented with RS, human intestinal organoids were formed.
(2) RS 2.5 μM group (the control group): When crypts were cultured in the basal media supplemented with RS 2.5 μM, human intestinal organoids were formed. Comparing the quantity of organoid calculated from the confluency, there is no significant difference with that in the RS 0 μM group.
(3) RS 5, 50 or 100 μM group (the experimental group according to the present invention): When crypts were cultured in the basal media supplemented with RS-246204 (5, 50 or 100 μM), human intestinal organoids were formed. Comparing the quantity of organoid calculated from the confluency, there is significant difference with that in the RS 0 or 2.5 μM groups (the control groups).

### Example 8. Formation of Organoids

Large intestinal tissue, adenoid tissue, and pancreatic cancer tissue were isolated from a patient during surgery and used in experiments for preparing and culturing organoids. Salivary tissue was isolated from a mouse and used in experiments for preparing and culturing organoids.

Each of the tissues was cut longitudinally from a proximal end to a distal end, and also laterally cut into pieces of about 5 mm length. The piece of the tissues obtained was washed with ice-cooled Dulbecco's phosphate-buffered saline (DPBS) until the supernatant liquid was sufficiently clear. Then, the tissues were isolated by treating with a Gentle Cell Dissociation Reagent (StemCell Technologies, Cambridge, MA), and filtering with a cell strainer. The isolated tissues were mixed in collagen gel, and placed in each well of a well plate. After keeping in 37° C. humidification incubator (5% CO₂), the cultured organoids were observed, and optical microscope photos were taken for analysis.

### Results:

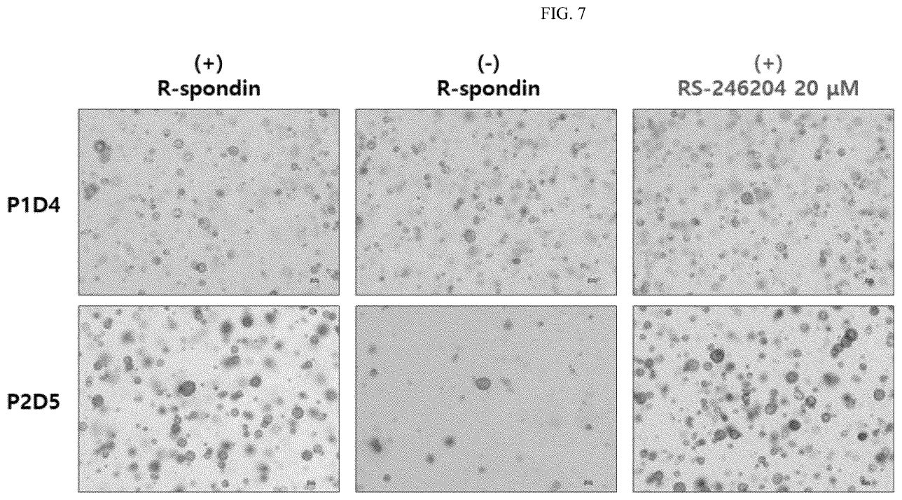

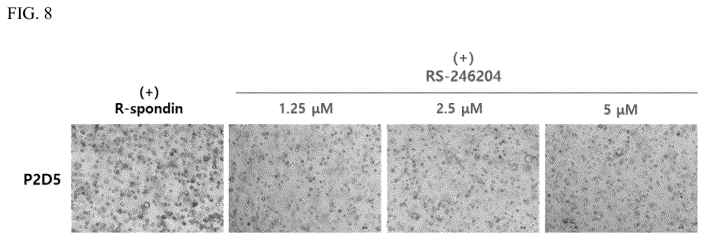

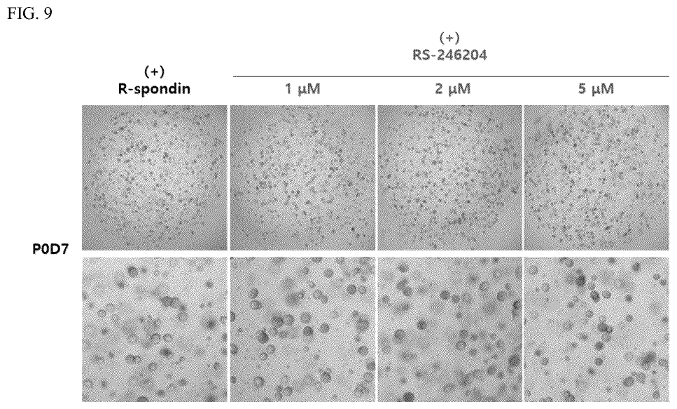

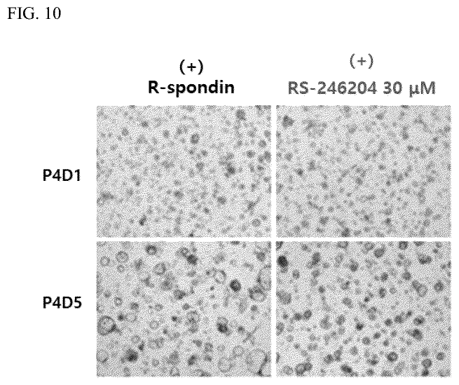

Large intestinal organoids, salivary organoids, adenoid organoids, and pancreatic cancer organoids obtained by culturing using the compound of the chemical formula 1 (namely, RS-246204) have characteristics below (see FIGS. 7 to 10).
(1) All kinds of organoid cultured using RS-246204 shows excellent formation and proliferation effects.
(2) The large intestinal organoids, the salivary organoids, and the pancreatic cancer organoids can grow even after subculturing.
(3) The large intestinal organoids grow while forming budding (crypt).
(4) RS-246204 makes the salivary organoids and the adenoid organoids form and proliferate in a dose-dependent manner.

### Example 9. Prophetic Example—Formation of Various Cancer Organoids Using RS-246204

Various types of tumor tissues, including pancreatic cancer, colorectal cancer, gastric cancer, liver cancer, lung cancer, breast cancer, prostate cancer, ovarian cancer, bladder cancer, esophageal cancer, glioblastoma, melanoma, thyroid cancer, renal cell carcinoma, cholangiocarcinoma, cervical cancer, endometrial cancer, testicular cancer, leukemia, lymphoma, and multiple myeloma, are surgically obtained from human patients or established from biopsy specimens.

Each tumor tissue is mechanically and enzymatically dissociated using a Gentle Cell Dissociation Reagent (StemCell Technologies, Cambridge, MA), followed by filtration through a 70-μm cell strainer to isolate tumor-derived epithelial clusters or single cells. The isolated cells are then embedded in collagen gel and seeded into each well of a 24-well culture plate. A basal medium such as Advanced DMEM/F12 is supplemented with RS-246204 at a concentration of 50 μM and added to each well.

The culture is maintained at 37° C. in a humidified incubator with 5% CO₂ for 5 to 10 days. It is expected that, under these conditions, tumor-derived organoids (“cancer organoids”) will form, exhibiting morphological characteristics of the corresponding tumor type. Organoids derived from solid tumors such as pancreatic, colorectal, or lung cancer are predicted to form gland-like or spheroid structures, while hematological malignancy-derived organoids (e.g., lymphoma or multiple myeloma) are expected to form non-adherent cell clusters or micro-aggregates.

The organoids are expected to be viable and proliferative, and may express tumor-specific molecular markers such as KRAS, TP53, or HER2, depending on the tumor origin. In particular, it is anticipated that the presence of RS-246204 in the basal medium will be sufficient to support organoid formation and growth without the need for additional exogenous growth factors such as EGF, Noggin, or R-spondin1.

Accordingly, this example demonstrates that RS-246204 may be broadly applicable to the formation of cancer organoids across multiple tumor types, and may serve as a key component in cancer organoid culture systems for research, drug screening, or therapeutic development.

### Example 10. Prophetic Example—Formation of Various Organoids Using RS-246204

Tissue samples are obtained from human donors or animal models and used to generate organoids from a wide range of organs, including the stomach, small intestine, colon, liver, thyroid gland, lung, brain, pancreas, kidney, heart, retina, skin, testis, ovary, blood vessels, bladder, salivary gland, thymus, prostate, bone marrow, lymph node, skeletal muscle, esophagus, gallbladder, bile duct, nasal cavity, and cochlea.

Each tissue is processed by mechanical mincing followed by enzymatic dissociation using Gentle Cell Dissociation Reagent (StemCell Technologies, Cambridge, MA). The resulting cell suspension is filtered through a 70-μm strainer to isolate epithelial clusters or progenitor-rich cell populations. The cells are embedded in collagen gel or Matrigel and seeded into well plates. A basal medium such as Advanced DMEM/F12 is supplemented with RS-246204 at a final concentration of 25-50 μM.

Cultures are maintained at 37° C. in a humidified incubator with 5% CO₂. It is expected that, under these conditions, tissue-derived organoids (“non-cancer organoids”) will form within 3 to 7 days, depending on the tissue type. Organoids derived from epithelial organs such as the stomach, intestine, lung, kidney, and liver are predicted to exhibit cystic or budding morphologies, while neuronal or sensory organoids such as retinal or cochlear organoids are expected to develop neural rosette-like or layered structures. Vascular and muscle organoids are anticipated to form branching or contractile structures over time.

The resulting organoids are expected to maintain key morphological and molecular characteristics of their tissue of origin. For instance, thyroid organoids may express TSH receptor and thyroglobulin, while pancreatic organoids may express insulin or PDX1. Structural features such as luminal formation, apical-basal polarity, and cell-type heterogeneity are also expected to be observed. Moreover, RS-246204 is anticipated to support organoid viability and self-renewal without the need for additional exogenous growth factors such as EGF, Noggin, or R-spondin1.

This example demonstrates that RS-246204 may be broadly applicable to the generation and maintenance of organoids from diverse human or animal tissues, and thus may be useful for applications such as disease modeling, regenerative medicine, and compound screening across a variety of tissue types.

Those skilled in the art will recognize, or be able to ascertain using no more than routine experimentation, many equivalents to the specific embodiments of the present disclosure described herein. Such equivalents are intended to be encompassed by the following claims.

What is claimed is:

1. A method for forming an organoid, comprising:
culturing tissues or cells in a culture medium comprising a compound of the following chemical formula 1:

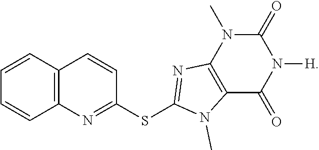

2. The method according to claim 1, wherein the organoid is cancer organoid or non-cancer organoid.

3. The method according to claim 1, wherein the organoid is derived from human or animal.

4. The method according to claim 1, wherein the organoid is used for transplantation into a subject for regenerative therapy.

5. The method according to claim 1, wherein the organoid is used for disease modeling, drug screening, or evaluation of efficacy or toxicity of a pharmaceutical, cosmetic, or functional food product.
